(19) United States
(12) Patent Application Publication
(10) Pub. No.: US 2025/0195182 A1
(43) Pub. Date: Jun. 19, 2025

(54) DEVICE AND METHOD FOR PROVIDING POWER TO SCANNER

(71) Applicant: MEDIT CORP., Seoul (KR)

(72) Inventors: Hyungsung KIM, Seoul (KR); Sungmun YOON, Seoul (KR)

(73) Assignee: MEDIT CORP., Seoul (KR)

(21) Appl. No.: 18/849,604

(22) PCT Filed: Mar. 23, 2023
(86) PCT No.: PCT/KR2023/003833

§ 371 Date: Sep. 23, 2024

(30) Foreign Application Priority Data
Mar. 25, 2022 (KR) 10-2022-0037632
Mar. 23, 2023 (KR) 10-2023-0037692

(51) Int. Cl. A61C 9/00; A61B 1/00; A61B 1/24; A61C 13/34; H02J 7/00

(52) U.S. Cl. CPC A61C 9/0053; A61B 1/00172; A61B 1/24; A61C 13/34; H02J 7/0013; H02J 7/0048; H02J 7/00712; H02J 7/007194; A61C 2204/002

(57) ABSTRACT

Provided is a scanner including a first insertion portion into which a battery is to inserted in a detachable form, a capacity indicating portion, and one or more processors configured to execute one or more instructions, wherein the one or more processors are configured to execute the one or more instructions to, in response to a first connector provided in the battery being connected to a second connector provided in the first insertion portion, receive battery information and power from the battery via the first connector and the second connector, and output an indicator corresponding to the battery information via the capacity indicating portion.

### TECHNICAL FIELD

An embodiment of the present disclosure relates to a device and method for supplying power, and more particularly, to a device and method for supplying power to a wireless scanner.

### BACKGROUND ART

Wireless scanners have been developed for convenience of use, portability, etc. Scanners process large volumes of data during scanning and have high power consumption. Therefore, when power is all consumed while a user is using a wireless scanner, problems such as powering off may occur. Accordingly, there is a need for a device and method for supplying power to a wireless scanner in order to enable the user to use the wireless scanner without interruption.

### DISCLOSURE

### Technical Solution

A scanner according to an embodiment may include a first insertion portion into which a battery is to inserted in a detachable form, a capacity indicating portion, and one or more processors configured to execute one or more instructions, wherein the one or more processors are configured to execute the one or more instructions to, in response to a first connector provided in the battery being connected to a second connector provided in the first insertion portion, receive battery information and power from the battery via the first connector and the second connector, and output an indicator corresponding to the battery information via the capacity indicating portion.

### DESCRIPTION OF DRAWINGS

FIG. 1 is a diagram to describe an image processing system according to an embodiment.

FIG. 2 is a block diagram of a data processing system according to an embodiment.

FIG. 3 illustrates a power supply device for a scanner, according to an embodiment.

FIG. 4 is a diagram illustrating a scanner according to an embodiment.

FIG. 5 is a diagram illustrating a battery being inserted into a scanner, according to an embodiment.

FIG. 6 is a diagram illustrating a charger according to an embodiment.

FIG. 7 is a diagram illustrating the interior of a charger according to an embodiment.

FIG. 8 is a graph showing a charge voltage and a charge current according to an embodiment.

FIG. 9 is graphs illustrating a charger adjusting a charge voltage and a charge current by taking into account a battery temperature, according to an embodiment.

FIG. 10 is a diagram illustrating a dummy cable being inserted into a scanner, according to an embodiment.

FIG. 11 is a power system diagram illustrating a power supply device for a scanner, according to an embodiment.

FIG. 12 is a diagram illustrating a data processing device outputting an interface screen showing a battery capacity, according to an embodiment.

FIG. 13 is a flowchart illustrating an operation in which a scanner receives power from a battery, according to an embodiment.

FIG. 14 is a flowchart illustrating an operation in which a charger charges a battery, according to an embodiment.

### MODE FOR INVENTION

In an embodiment, the battery information may include a battery capacity and a battery temperature, and the indicator corresponding to the battery information may indicate the battery capacity.

In an embodiment, the capacity indicating portion may include at least one light-emitting diode (LED) element, and the indicator corresponding to the battery information may indicate an interval corresponding to the battery information based on at least one of an on/off of the at least one LED element, a color of output light of the at least one LED element, and blinking of output light thereof.

In an embodiment, the battery may include at least one of one of a protrusion and a groove and a metal that adheres to a magnet, and the first insertion portion may include at least one of one of a groove and a protrusion for engaging with the protrusion or the groove in the battery and a magnet, and be coupled with the battery by using the at least one of the one of the groove and the protrusion and the magnet.

In an embodiment, a portion of the battery inserted into the first insertion portion and the first insertion portion may each include a round region and an angular region, and the first connector and the second connector are respectively provided in the angular regions of the portion of the battery and the first insertion portion.

In an embodiment, in response to a dummy cable, which is not the battery, being inserted into the first insertion portion, the scanner may receive power from a charger via the dummy cable, the dummy cable may include a dummy portion including a fourth connector and inserted into the first insertion portion and a connection portion connected to the charger, and the one or more processors may be further configured to execute the one or more instructions to receive power from the charger via the second connector and the fourth connector.

In an embodiment, the scanner may further include an optical unit, and the one or more processors may be further configured to execute the one or more instructions to, in response to receiving the power from the battery, control the optical unit to obtain scan data regarding an object, and generate a three-dimensional oral cavity model for the object based on the scan data regarding the object.

A charger according to an embodiment may charge a battery that is inserted into a scanner in a detachable form to supply power to the scanner, and may include a second insertion portion into which the battery is inserted and provided with a third connector, a charger integrated circuit (IC) chip connected to the third connector via a printed circuit board (PCB), and a state indicating portion configured to indicate a state of charge of the battery, wherein the charger IC chip is configured to, in response to the third connector being connected to a first connector of the battery, identify a battery capacity and a battery temperature via the third connector and the first connector, determine a charge voltage and a charge current based on the battery capacity and the battery temperature, charge the battery by providing the determined charge voltage and charge current to the battery via the third connector and the first connector, and indicate a state of charge of the battery via the state indicating portion.

In an embodiment, the state of charge of the battery may include at least one of a state in which the battery is not inserted, a state in which the battery is being charged, a state in which charging is complete, and a state in which an error has occurred.

In an embodiment, the second insertion portion may include a plurality of insertion spaces into which a plurality of batteries are to be independently inserted, the charger IC chip may be included as a plurality of charger IC chips, wherein the number of charger IC chips is equal to the number of the plurality of insertion spaces, to control charging of the plurality of batteries respectively inserted into the plurality of insertion spaces, and the state indicating portion may include a plurality of LEDs in a number that is as many as the number of the plurality of insertion spaces, each of the plurality of LED elements independently indicating a state of charge of each of the plurality of batteries respectively inserted into the plurality of insertion spaces.

In an embodiment, a first charger IC chip among the plurality of charger IC chips may be configured to identify a first battery capacity and a first battery temperature for a first battery inserted into a first insertion space among the plurality of insertion spaces, determine, based on the first battery capacity and the first battery temperature, a first charge voltage and a first charge current to be provided to the first battery, and provide the determined first charge voltage and first charge current to the first battery.

In an embodiment, the charger may further include a connection port, and in response to the other end of a dummy cable, of which one end is inserted into the scanner, being connected to the connection port, the charger may supply power to the scanner via the dummy cable.

A battery that is rechargeable according to an embodiment may be inserted into a scanner in a detachable form to provide power to the scanner, and include a first connector, and an IC chip, wherein the IC chip may be configured to obtain battery information including data indicating a battery capacity and data indicating a battery temperature, and in response to the first connector being connected to a second connector provided in the scanner, transmit power and the battery information to the scanner via the first connector and the second connector.

In an embodiment, in response to the battery being inserted into a second insertion portion included in a charger and the first connector being connected to a third connector provided in the second insertion portion, the IC chip may be configured to transmit the battery temperature to the charger via the first connector and the third connector, and the battery is charged by receiving power from the charger.

In an embodiment, the battery may include at least one of one of a protrusion and a groove and a metal that adheres to a magnet, at least one of the scanner and the charger may include at least one of one of a groove and a protrusion for engaging with the protrusion or the groove in the battery and a magnet, and the battery may be mounted to the scanner or the charger by using the at least one of the one of the protrusion and the groove and the metal that adheres to the magnet.

An operation method performed by a scanner into which a battery is to be inserted in a detachable form may include receiving battery information and power from a battery in response to a connector of the battery being connected to a connector of the scanner, and outputting an indicator corresponding to the battery information.

In an embodiment, the battery information may include a battery capacity and a battery temperature, and the indicator corresponding to the battery information may indicate the battery capacity.

An operation method of a charger for charging a battery that is inserted into a scanner in a detachable form and supplies power to the scanner may include identifying a battery capacity and a battery temperature in response to a connector of the charger being connected to a connector of the battery, determining a charge voltage and a charge current based on the battery capacity and the battery temperature, charging the battery by providing the determined charge voltage and charge current to the battery, and indicating a state of charge of the battery.

A computer-readable recording medium according to an embodiment may be a recording medium having recorded thereon a program for implementing an operation method performed by a scanner, the operation method including receiving battery information and power from a battery in response to a connector of the battery being connected to a connector of the scanner, and outputting an indicator corresponding to the battery information.

A computer-readable recording medium according to an embodiment may be a recording medium having recorded thereon a program for implementing an operation method of a charger for charging a battery supplying power to a scanner, the operation method including identifying a battery capacity and a battery temperature in response to connectors of the charger and the battery being connected to each other, determining a charge voltage and a charge current based on the battery capacity and the battery temperature, charging the battery by providing the determined charge voltage and charge current to the battery, and indicating a state of charge of the battery.

The present specification describes principles of the present application and sets forth embodiments to clarify the scope of the present application and to allow one of ordinary skill in the art to implement the present application. The embodiments set forth herein may be implemented in various forms.

Like reference numerals denote like elements throughout. The present specification does not describe all components in the embodiments, and common knowledge in the art or the same descriptions of the embodiments will be omitted below. Terms such as ‘part’ and ‘portion’ used herein denote those that may be embodied using software or hardware, and according to embodiments, a plurality of ‘parts’ or ‘portions’ may be embodied as a single unit or element, or a single ‘part’ or ‘portion’ may include a plurality of units or elements. Hereinafter, the operating principles and embodiments of the present application are described in detail with reference to the accompanying drawings.

Furthermore, in this specification, raw data or the like may be obtained using at least one camera to represent an object in two or three dimensions. Specifically, the raw data is data obtained to generate an image of the object, and may be data (e.g., two-dimensional (2D) data) obtained from at least one image sensor included in a three-dimensional (3D) scanner when scanning the object by using the 3D scanner. The raw data may be 2D image data or 3D image data.

In addition, wireless scanners are being developed for convenience of use, portability, etc. Wireless scanners may receive power via batteries.

Scanners process large volumes of data during scanning and have high power consumption. Therefore, when power is all consumed while a user is using a wireless scanner, problems such as powering off may occur. Accordingly, there is a need for a device and method for supplying power to a wireless scanner to enable the user to use the wireless scanner without interruption.

The disclosed embodiment is intended to address the need for the above-described technology and provide a power supply device and a power supply method for supplying power to a scanner. Furthermore, the disclosed embodiments are intended to provide a charger and a charging method capable of charging a battery that is inserted into a scanner in a detachable form.

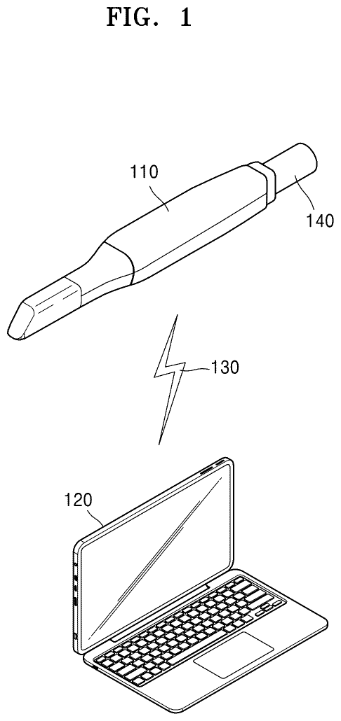

Hereinafter, embodiments are described in detail with reference to the drawings. FIG. 1 is a diagram to describe an image processing system according to an embodiment.

Referring to FIG. 1, the image processing system may include a scanner 110 and a data processing device 120 coupled to the scanner 110 via a communication network 130.

The scanner 110 may be a medical device that obtains an image of an object.

In the present disclosure, the object may refer to a target to be scanned. The object may include a part of a body or a model that mimics the part of the body. The object may include at least one of an oral cavity, an ear, a nose, an artificial structure, or a plaster model that mimics the oral cavity, ear, nose, or artificial structure. For example, the object may include at least one of a tooth and a gingiva or a plaster model or impression model of at least one of a tooth and a gingiva, and/or an artificial structure insertable into an oral cavity or a plaster model or impression model of such an artificial structure. In this case, the artificial structure insertable into the oral cavity may include, for example, at least one of an orthodontic appliance, an implant, a crown, an inlay, an onlay, an artificial tooth, and an orthodontic auxiliary device inserted into the oral cavity. In addition, the orthodontic appliance may include at least one of a bracket, an attachment, an orthodontic screw, a lingual orthodontic appliance, and a removable orthodontic retainer.

The scanner 110 may obtain an image of an object by scanning at least one of an oral cavity, an ear, a nose, an artificial structure, or a plaster model that mimics the oral cavity, the ear, the nose, or the artificial structure. The image may also be referred to as a frame.

In an embodiment, the scanner 110 may be a wireless scanner. In an embodiment, the scanner 110 may wirelessly transmit and receive data to and from the data processing device 120.

In an embodiment, a battery 140 may be inserted into the scanner 110 in a detachable form. The battery 140 may be inserted entirely into the scanner 110, but is not limited thereto, and as shown in FIG. 1, only a region of the battery 140 may be inserted into the scanner 110 and the remaining region may protrude out of the scanner 110.

The scanner 110 may operate by receiving power from the battery 140, in response to the battery 140 being inserted. In an embodiment, the battery 140 that supplies power to the scanner 110 may be a rechargeable battery.

In an embodiment, the battery 140 may be inserted into the scanner 110 and transmit battery information to the scanner 110. In an embodiment, the battery information may include at least one of data indicating a battery capacity and data indicating a battery temperature. In an embodiment, the data indicating the battery capacity may include at least one of the maximum capacity of the battery 140 and a current charge capacity of the battery 140.

In an embodiment, the scanner 110 may receive the battery information from the battery 140 and identify the battery capacity based on the battery information. In an embodiment, the scanner 110 may output an indicator corresponding to the battery capacity. A user may easily identify the battery capacity by using the indicator output by the scanner 110.

In an embodiment, the scanner 110 may operate by receiving power from the battery 140, but is not limited thereto, and the scanner 110 may also receive power via a wire. For example, the scanner 110 may be connected to a power adapter or a battery charger via a wired cable and receive power from the power adapter or the battery charger via a wire.

In an embodiment, the scanner 110 may be powered on in response to receiving power via the battery 140 or the wired cable, and may perform a scan operation on the object.

In an embodiment, the scanner 110 may be a handheld scanner that is held in a user's hand and moved to scan an object.

The scanner 110 may be inserted into the ear or nose and scan the ear or nose in a non-contact manner to obtain an image of the ear or nose. Alternatively, the scanner 110 may be an intraoral scanner that obtains an intraoral image of the oral cavity including at least one tooth by being inserted into the oral cavity and scanning teeth.

Hereinafter, for convenience of description, a case in which the scanner 110 is an intraoral scanner is described as an example, but the present disclosure is not limited thereto.

The scanner 110 may include a main body and a tip. The main body may include a light irradiator that projects light and a camera that obtains an image by capturing an image of an object.

The tip is a part that is inserted into the oral cavity and may be attached to the main body with a detachable structure. The tip may include a light path changing member to guide light irradiated from the main body toward the object and guide light received from the object toward the main body.

The scanner 110 may obtain an image of the object by scanning the object.

In an embodiment, the image may include an image showing at least one tooth, an oral cavity including the at least one tooth, or a plaster model of the oral cavity (hereinafter, an “intraoral image”).

In an embodiment, the image of the object may include a 2D image of the object or a 3D intraoral image representing the object in a 3D manner. The 3D intraoral image may be generated by modeling a structure of the oral cavity in three dimensions based on raw data, and thus may also be referred to as a ‘3D intraoral model’. The 3D intraoral image may also be referred to as a 3D scan model or 3D scan data.

The scanner 110 may obtain surface information about the object as raw data in order to image a surface of at least one of a tooth and a gingiva in the oral cavity and an artificial structure insertable into the oral cavity (e.g., an orthodontic appliance including brackets, wires, etc., an implant, an artificial tooth, an orthodontic auxiliary device inserted into the oral cavity, etc.).

The scanner 110 may obtain surface information about the object as raw data in order to image a surface of at least one of a tooth or a gingiva in the oral cavity, an artificial structure insertable into the oral cavity (e.g., an orthodontic appliance including brackets, wires, etc., an implant, an artificial teeth, an orthodontic auxiliary device inserted into the oral cavity, etc.), and a model such as a plaster model or impression model, a dentiform model, or the like.

The scanner 110 may transmit the obtained raw data to the data processing device 120 via the communication network 130.

In addition, in an embodiment, the scanner 110 may transmit data indicating a battery capacity to the data processing device 120.

The data processing device 120 may be connected to the scanner 110 via a wired or wireless communication network 130. For example, the data processing device 120 may be connected to the scanner 110 via a wireless hub. The wireless hub may be a wireless device that operates via a wireless module.

The data processing device 120 may be any electronic device capable of receiving raw data from the scanner 110, generating an intraoral image based on the received raw data, and processing, displaying, and/or transmitting the intraoral image. For example, the data processing device 120 may be a computing device such as a smartphone, a laptop computer, a desktop computer, a personal digital assistant (PDA), a tablet PC, etc., but is not limited thereto. Furthermore, the data processing device 120 may exist in the form of a server (or a server apparatus) for processing an intraoral image. The data processing device 120 may generate a 3D intraoral image, i.e., 3D scan data, based on the raw data received from the scanner 110. The data processing device 120 may display the 3D intraoral image on a display, or output or transmit it to an external device.

In another example, the scanner 110 may obtain raw data through intraoral scanning, process the obtained raw data to generate 3D information, and transmit the 3D information to the data processing device 120.

In an embodiment, the scanner 110 may obtain 3D information about the object by using various methods. For example, the scanner 110 may obtain 3D information about the object by using a confocal method. Alternatively, in an embodiment, the scanner 110 may obtain 3D information about the object by using an optical triangulation technique. However, this is an embodiment, and the scanner 110 may obtain 3D information from the raw data by using various methods other than the confocal method or the optical triangulation technique, and transmit the 3D information to the data processing device 120.

The data processing device 120 may analyze, process, and display the received 3D information and/or transmit it to an external device.

The data processing device 120 may generate a 3D oral cavity model for the object by using the image, i.e., the frame, of the object received from the scanner 110.

In an embodiment, the data processing device 120 may receive data indicating a battery capacity from the scanner 110. In an embodiment, in response to receiving the data indicating the battery capacity, the data processing device 110 may output an interface screen indicating the battery capacity via the display. The user may easily identify a charge capacity of the battery 140 in response to the data processing device 120 outputting the interface screen indicating the battery capacity via the display.

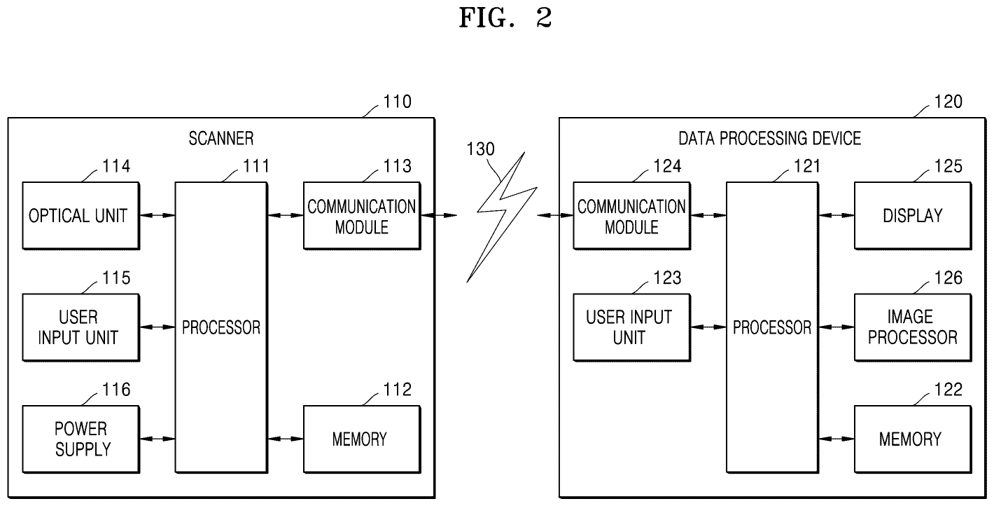

FIG. 2 is a block diagram of a data processing system according to an embodiment.

In an embodiment, the data processing system may include a scanner 110, a data processing device 120, and a communication network 130.

The scanner 110 may obtain raw data by scanning the object. The object may include, for example, a cast model of a patient's oral cavity or teeth, but is not limited thereto. The scanner 110 may transmit the obtained raw data to the data processing device 120 via the communication network 130, or may process the raw data to generate a 3D virtual model and transmit the 3D virtual model to the data processing device 120.

The scanner 110 may include a processor 111, a memory 112, a communication module 113, an optical unit 114, a user input unit 115, and a power supply 116.

The memory 112 may store at least one instruction. Furthermore, the memory 112 may store at least one instruction executed by the processor 111. In addition, the memory 140 may store at least one program executed by the processor 111.

The communication module 113 may perform communication with the data processing device 120 via the communication network 130 that is wired or wireless.

In an embodiment, the scanner 110 and the data processing device 120 may be connected via a wireless hub. To this end, the communication module 113 of the scanner 110 may include a transmitter, and the wireless hub may include a receiver. The wireless hub may be connected to the data processing device 120 via a cable.

In an embodiment, the communication module 113 may transmit and receive control signals to and from the data processing device 120. Furthermore, the communication module 113 may transmit information about an operating status of the scanner 110 to the data processing device 120. Furthermore, the communication module 113 may transmit raw data obtained by the optical unit 114 to the data processing device 120.

In an embodiment, the communication module 113 may transmit data indicating a battery capacity to the data processing device 120.

The communication interface 110 may include at least one short-range communication module that performs communication according to a communication standard such as Bluetooth, Wi-Fi, Bluetooth Low Energy (BLE), near field communication (NFC)/radio frequency identification (RFID), Wi-Fi Direct, ultra-wideband (UWB), ZigBee, or the like, a long-range communication module that communicates with a server for supporting long-range communication according to long-range communication standards, and at least one port for connecting to an external electronic device via a wired cable in order to communicate with the external electronic device in a wired manner.

The optical unit 114 may include an optical module and a projector. The optical unit 114 may include a light source, a projector that projects light from the light source, and at least one camera that receives light reflected from the object. The optical unit 114 may project patterned light or structured light. The optical unit 114 may irradiate light by using a light source and control each of micro-mirrors included in a digital micromirror to form a pattern. The optical unit 114 may irradiate light by controlling the mirrors included in the DMD to turn on or off. The optical unit 114 may irradiate the object with light, and obtain 3D data representing the shape of the object by scanning the object irradiated with the light.

The user input unit 115 may receive a user input for controlling the scanner 110. The user input unit 115 may also be referred to as a user interface.

The user input unit 115 may include a touch panel for sensing a user's touch, a button for receiving a user's push manipulation, a voice recognition device including a microphone, etc. Alternatively, the user input unit 115 may further include at least one of a wheel or dome switch for receiving a user's rotation manipulation and a sensor (not shown) capable of recognizing a motion.

According to an embodiment, the power supply 116 may receive power and provide it to each component of the scanner 110.

In an embodiment, the power supply 116 may include a space into which the battery 140 may be inserted.

In an embodiment, the battery 140 may be provided with a connector. In an embodiment, the power supply 116 of the scanner 110 may also be provided with a connector. In an embodiment, in response to the connector of the power supply 116 being connected to the connector of the battery 140, the power supply 116 may receive power from the battery 140.

In an embodiment, the power supply 116 may include a capacity indicating portion. In an embodiment, the capacity indicating portion may include at least one LED element. In an embodiment, the power supply 116 may output an indicator via the capacity indicating portion.

In an embodiment, the power supply 116 may output an indicator corresponding to battery capacity information based on the battery capacity information. The indicator corresponding to the battery capacity information may be information indicating a level of the battery capacity. The power supply 116 may output the indicator corresponding to the battery capacity information as at least one of an on/off of the at least one LED element, an output color of the LED element, and blinking of the LED element.

In an embodiment, the power supply 116 may include a secondary battery or capacitor for storing energy. In an embodiment, when the power supply 116 includes a secondary battery or capacitor, the capacitor or the like may store power received from the battery 140 or the like as electrical energy. The secondary battery or capacitor may store electrical energy and then provide the electrical energy to the scanner 110 so that the scanner 110 does not immediately turn off when the battery 140 is completely discharged while the scanner 110 is operating.

The processor 111 may control all operations of the scanner 110. The processor 111 may control at least one component included within the scanner so that an intended operation is performed. Thus, even when it is described that the processor 111 performs certain operations, it may be understood that the processor 111 controls at least one component included in the scanner 110 so that the certain operations are performed. The processor 111 may control the optical unit 214 to obtain 3D data regarding the object.

In an embodiment, the processor 111 may receive power from the battery 140 in response to the battery 140 being inserted into the power supply 116, and control the components in the scanner 110 based on receiving power.

In an embodiment, the processor 111 may turn on the scanner 110 when the user selects a power button included in the user input unit 115 while power is supplied via the power supply 116 so that the scanner 110 may operate.

In an embodiment, the processor 111 may control the power supply 116 to receive battery information from the battery 140 and output an indicator corresponding to a battery capacity based on the battery information.

Hereinafter, the data processing device 120 is described. The data processing device 120 may also be referred to as an intraoral image processing device. The data processing device 120 may include a processor 121, a memory 122, a user input unit 123, a communication module 124, a display 125, and an image processor 126.

The user input unit 123 may receive a user input for controlling the data processing device 120. The user input unit 123 may include a user input device including a touch panel for sensing a user's touch, a button for receiving a user's push manipulation, a mouse or keyboard for specifying or selecting a point on a screen of the user input unit, etc., and may include a voice recognition device for voice recognition or a motion sensor for motion recognition.

The communication module 124 may perform communication with at least one external electronic device via a wired or wireless communication network. The communication module 124 may perform communication with the scanner 110 according to control by the processor 121.

In detail, the communication module 124 may include at least one short-range communication module that performs communication according to a communication standard such as Bluetooth, Wi-Fi, BLE, NFC/RFID, Wi-Fi Direct, UWB, ZigBee, or the like. Furthermore, the communication module 124 may further include a long-range communication module that communicates with a server for supporting long-range communication according to long-range communication standards.

In addition, the communication module 124 may include at least one port for connecting to an external electronic device, such as the scanner 110 or the like, via a wired cable.

In an embodiment, the communication module 124 may transmit a control signal to the scanner 110. The control signal transmitted to the scanner 110 may include at least one of a power-on command or a power-off command for the scanner 110, a command for the scanner 110 to enter a scan mode, or a command for the scanner 110 to enter a standby mode.

In an embodiment, the communication module 124 may receive data indicating a battery capacity from the scanner 110.

The display 125 may display a certain screen according to control by the processor 121. The display 125 may output a user interface screen for performing user input. The display 125 may display a user interface screen including an intraoral image generated based on data obtained by scanning a patient's oral cavity or a plaster model of the oral cavity via the scanner 110. In addition, the display 125 may output a 3D oral cavity model generated based on 2D image data received from the scanner 110.

In an embodiment, the display 125 may output an interface screen indicating the battery capacity. In an embodiment, the interface screen indicating the battery capacity may include information indicating a current charge capacity of the battery 140 relative to the maximum charge capacity of the battery 140 by using pictures, numbers, letters, colors, etc. The user may easily identify the charge capacity of the battery 140 by viewing the interface screen indicating the battery capacity output on a screen of the data processing device 120.

The image processor 126 may perform operations for generating and/or processing images. In detail, the image processor 126 may receive raw data obtained from the scanner 110 and generate a 3D virtual model based on the received data.

The memory 122 may store at least one instruction. Furthermore, the memory 140 may store at least one instruction executed by the processor 121. Furthermore, the memory 122 may store at least one program executed by the processor 121. In addition, the memory 122 may store data received from the scanner 110 (e.g., raw data obtained through intraoral scanning, etc.). Alternatively, the memory 122 may store an intraoral image representing an oral cavity in three dimensions. According to an embodiment, the memory 122 may include one or more instructions for obtaining a 3D oral cavity model from 2D image data.

The processor 121 controls an intended operation to be performed by executing at least one instruction stored in the memory 122. In this case, the at least one instruction may be stored in an internal memory included in the processor 121 or in the memory 122 included in the data processing device 120 separately from the processor 121.

In an embodiment, the processor 121 may transmit a control signal to the scanner 110 by executing one or more instructions stored in the memory 122, so that the scanner 110 is controlled according to the control signal.

According to an embodiment, performing, by the processor 121, operations such as ‘extraction’, ‘obtaining’, ‘generation’, etc. may include not only a case where the processor 121 directly performs the above-described operations by executing at least one instruction but also a case where the processor 121 controls other components to perform the above-described operations.

In order to implement the embodiments presented in the present disclosure, the scanner 110 and the data processing device 120 may include only some of the components shown in FIG. 2 or include more components than those shown in FIG. 2.

Furthermore, the data processing device 120 may store and execute dedicated software linked to the scanner 110. Here, the dedicated software may be referred to as a dedicated program, a dedicated tool, or a dedicated application. When the data processing device 120 operates in conjunction with the scanner 110, the dedicated software stored in the data processing device 120 may be coupled to the scanner 110 to receive in real time data obtained through intraoral scanning.

In addition, the dedicated software may transmit and receive control signals to and from the scanner 110, and may also perform at least one operation for obtaining, processing, storing, and/or transmitting an intraoral image. Here, the dedicated software may be stored in the processor 121.

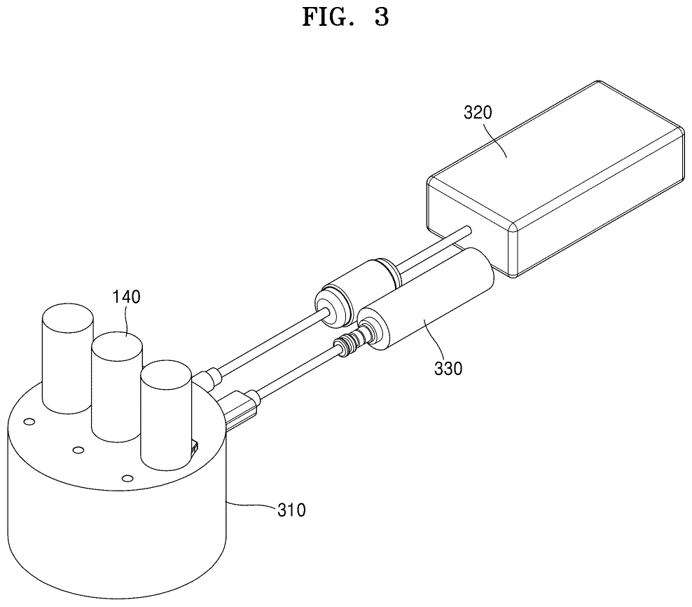

FIG. 3 illustrates a power supply device for a scanner, according to an embodiment.

Referring to FIG. 3, a power supply device for a scanner according to an embodiment may include a charger 310, a battery 140, an adapter 320, and a dummy cable 330.

In an embodiment, the power supply device for the scanner may be a device that supplies power to the scanner 110. In an embodiment, the scanner 110 may be supplied with power from the battery 140 that is charged, or may be supplied with power via the dummy cable 330.

First, a case where the scanner 110 is supplied with power from the battery 140 is described.

In an embodiment, the battery 140, the scanner 110, and the charger 310 may each be provided with a connector.

In an embodiment, the battery 140 may obtain battery information. In an embodiment, the battery information may include at least one of data indicating a battery capacity and data indicating a battery temperature.

In an embodiment, the battery 140 may be a rechargeable battery. In an embodiment, the battery 140 may be charged by the charger 310.

In an embodiment, the charger 310 may include an insertion portion into which the battery 140 may be inserted. In an embodiment, the charger 310 may include a plurality of insertion portions into which a plurality of batteries 140 may be inserted.

The charger 310 may be connected to the adapter 320 via a connection cable. The adapter 320 is a device that converts an alternating current from a power system into a direct current, and may supply power to the charger 310.

In an embodiment, when the battery 140 is inserted into the charger 310 so the connector provided in the charger 310 is connected to the connector provided in the battery 140, the charger 310 may identify a battery capacity and a battery temperature. In an embodiment, the charger 310 may determine a charge voltage and a charge current, based on the battery capacity and the battery temperature. In an embodiment, the charger 310 may charge the battery 140 by providing the determined charge voltage and charge current to the battery 140.

In an embodiment, the charger 310 may indicate a state of charge of the battery 140. In an embodiment, the charger 310 may include an LED element for indicating the state of charge of the battery 140. In an embodiment, the charger 310 may include a plurality of LED elements each indicating the state of charge of each of the plurality of batteries 140. In an embodiment, the state of charge of the battery 140 may include at least one of a state in which the battery 140 is not inserted, a state in which the battery 140 is being charged, a state in which charging is complete, and a state in which an error has occurred.

In an embodiment, the battery 140 that is charged may be inserted into the scanner 110. In an embodiment, when the battery 140 is inserted into the scanner 110 so the connector provided in the scanner 110 is connected to the connector provided in the battery 140, the battery 140 may transmit battery information to the scanner 110. Furthermore, the battery 140 may supply power to the scanner 110 via the connector.

Next, a case where the scanner 110 is supplied with power via the dummy cable 330 is described.

In an embodiment, the dummy cable 330 may be a power supply device in the form of a cable used instead of the battery 140. The dummy cable 330 does not require charging because the dummy cable 330 supplies power to the scanner 110 via a cable. The dummy cable 330 may include a dummy portion having the shape of a battery and a connection portion connected to the charger 310. The dummy portion of the dummy cable 330 may have the same shape as the battery 140 that is inserted into the scanner 110. The dummy portion may be provided with a connector to connect with the connector of the scanner 110.

In an embodiment, the dummy cable 330 may receive power from the charger 310 and provide the received power to the scanner 140 via the connector provided in the dummy portion.

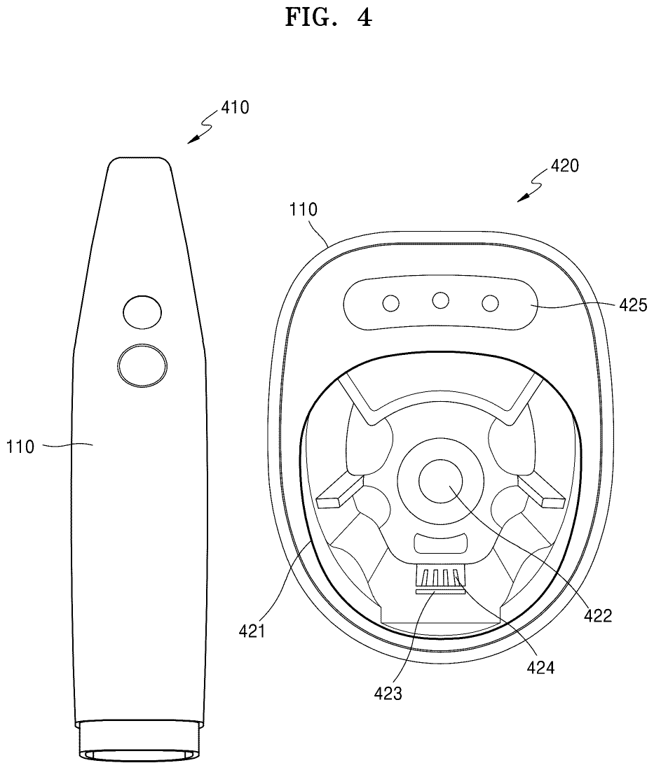

FIG. 4 is a diagram illustrating a scanner 110 according to an embodiment.

In FIG. 4, the drawing reference numeral 410 represents a drawing showing the front of a main body of the scanner 110. In an embodiment, the scanner 110 may include a tip and the main body. Hereinafter, for convenience of description, both a scanner main body without a tip and a scanner main body with the tip attached thereto are referred to as the scanner 110.

In an embodiment, a frame or a case enclosing the front of the scanner 110 may include a user input unit. The user input unit may include, for example, a button or the like, and may receive a user input for controlling the scanner 110 and/or the data processing device 120 connected to the scanner 110.

In FIG. 4, the drawing reference numeral 420 represents a bottom view of the scanner 110 as viewed from the bottom. In an embodiment, a space into which the battery 140 may be inserted may be formed at a bottom of the scanner 110. In an embodiment, the space formed in the scanner 110 and into which the battery 140 may be inserted is referred to as a first insertion portion 421. The first insertion portion 421 may be the space having a concave shape with a center portion recessed from the bottom of the scanner 110.

In an embodiment, a region of the battery 140 may be inserted into the first insertion portion 421. In an embodiment, the region of the battery 140 that is inserted into the first insertion portion 421 is referred to as a first region.

In an embodiment, to facilitate insertion of the first region of the battery 140 into the first insertion portion 421, the first insertion portion 421 and the first region of the battery 140 may have shapes corresponding to each other. For example, an innermost region of the first insertion portion 421 that is concavely recessed may have the same shape as one side of the first region of the battery 140. For example, when one end of the one side of the first region of the battery 140 has a flat, angular shape and the remaining portion thereof has a round shape, as shown in FIG. 4, the innermost region of the first insertion portion 421 may also be formed to have one end of a flat shape and the remaining portion of a round shape.

In an embodiment, a magnet 422 may be provided in the innermost region of the first insertion portion 421 that is concavely recessed. In an embodiment, a metal may be provided on the one side of the first region of the battery 140. The metal provided on the one side of the first region of the battery 140 may be a metal that adheres to the magnet. In an embodiment, the magnet 422 included in the first insertion portion 421 may be combined with the metal provided on the one side of the first region of the battery 140, thereby allowing the battery 140 to be easily attached to the scanner 110.

In an embodiment, the first insertion portion 421 may include one of a protrusion and a groove. For example, FIG. 4 illustrates a case where a groove 423 is formed in the first insertion portion 421. The groove 423 provided in the first insertion portion 421 may be used to engage with a protrusion provided in the first region of the battery 140.

In an embodiment, the first insertion portion 421 may include a terminal. In an embodiment, the terminal provided in the first insertion portion 421 is referred to as a second connector 424. In an embodiment, the second connector 424 may be formed in a flat region of the first insertion portion 421.

In an embodiment, the second connector 424 is connected to a connector provided in the battery 140 or the dummy cable 330 to receive power from the battery 140 or the dummy cable 330.

In an embodiment, the scanner 110 may include a capacity indicating portion 425. In an embodiment, the capacity indicating portion 425 may include at least one LED element. In an embodiment, the capacity indicating portion 425 may display a capacity of the battery 140 inserted into the first insertion portion 421 by using the at least one LED element.

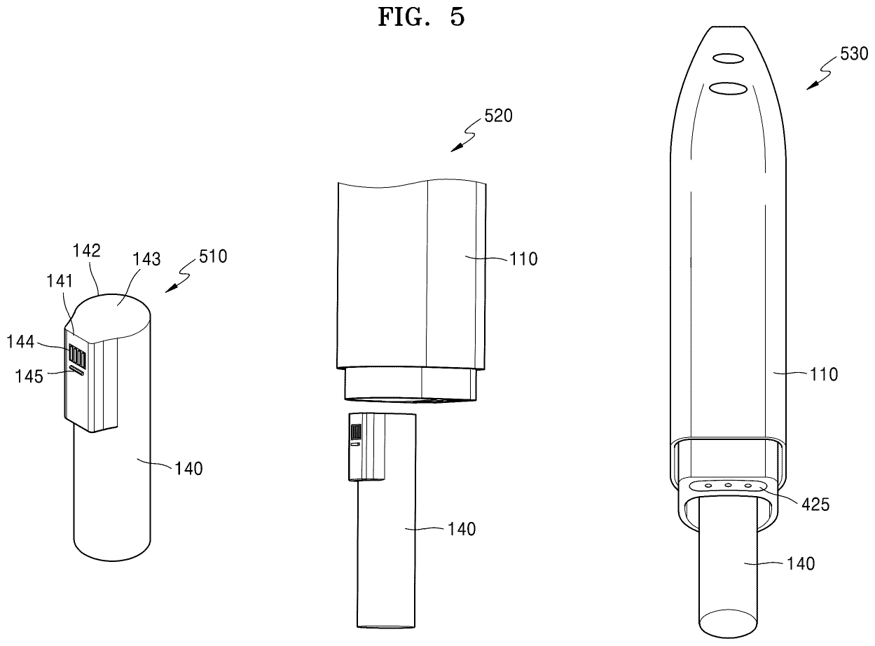

FIG. 5 is a diagram illustrating the battery 140 being inserted into the scanner 110, according to an embodiment.

In FIG. 5, the drawing reference numeral 510 illustrates the battery 140. In an embodiment, the battery 140 may be a rechargeable battery that is inserted into the scanner 110 in a detachable form to provide power to the scanner 110.

In an embodiment, a first region of the battery 140 may be inserted into the first insertion portion formed in the scanner 110. In an embodiment, the first region may be the entire region of the battery 140. For example, the entire battery 140 may be inserted and installed into the first insertion portion without any region of the battery 140 protruding out of the scanner 110. Alternatively, the first region may be a portion of the entire region of the battery 140. In this case, the first region of the battery 140 may be inserted into the first insertion portion, and the remaining region other than the first region may be mounted in the scanner 110 while protruding out of the scanner 110.

In an embodiment, the first region of the battery 140 may have a shape corresponding to the first insertion portion 421. For example, as described with reference to FIG. 4, when one end of the innermost region of the first insertion portion that is concavely recessed has an angular and flat shape, and the remaining portion thereof has a round shape, one end 141 of the first region of the battery 140 may also be formed to have an angular and flat shape and the remaining portion 142 to have a round shape.

In an embodiment, a metal that adheres to a magnet may be provided on one side 143 of the first region of the battery 140.

In an embodiment, the metal provided on the one side 143 of the first region of the battery 140 may be attached to the magnet 422 included in the first insertion portion provided in the scanner 110, thereby allowing the battery 140 to be fixed to the scanner 110.

In an embodiment, the first region of the battery 140 may include one of a protrusion and a groove. For example, as described with reference to FIG. 4, when the groove 423 is formed in the first insertion portion of the scanner 110, a protrusion may be formed in the first region of the battery 140 and may engage with the groove 423 formed in the first insertion portion of the scanner 110.

In an embodiment, the first region of the battery 140 may include a terminal for electrically connecting with another electronic device. In an embodiment, the terminal provided in the first region of the battery 140 may be referred to as a first connector 144. In an embodiment, the first connector 144 may be formed on a flat portion of the first region.

In an embodiment, the first connector 144 may be electrically connected to a connector provided in the scanner 110 or the charger 310.

In an embodiment, the battery 140 may include an IC chip. In an embodiment, the IC chip included in the battery 140 may obtain battery information. The battery information may include at least one of data indicating a battery capacity and data indicating a battery temperature.

In an embodiment, a temperature sensor may be provided inside the battery 140. The IC chip included in the battery 140 may transmit a temperature of the battery, which is sensed by the temperature sensor, to the connector provided in the scanner 110 or charger 310 via the first connector 144.

In FIG. 5, the drawing reference numeral 520 illustrates the battery 140 being inserted into the first insertion portion formed at the bottom of the scanner 110.

In FIG. 5, the drawing reference numeral 530 illustrates a case where the battery 140 is inserted and installed into the first insertion portion formed at the bottom of the scanner 110. In an embodiment, the battery 140 may be inserted entirely into the scanner 110, but is not limited thereto, and only a region of the battery 140 may be inserted into the scanner 110 and the remaining region may protrude out of the scanner 110.

FIG. 5 illustrates the remaining region of the battery 140 protruding out of the scanner 110. The user may easily separate the battery 140 from the scanner 110 by using the remaining region of the battery 140 that protrudes out of the scanner 110.

The scanner 110 may be turned off when power is not supplied thereto. When the power is turned off while the user is performing a scan by using the scanner 110, problems such as loss of data obtained by the user through the scan may occur. In addition, user convenience may be reduced.

In an embodiment, the scanner 110 may display a battery capacity by using the capacity indicating portion 425. In an embodiment, the capacity indicating portion 425 may include one or more LED elements. In an embodiment, the capacity indicating portion 425 may output an indicator corresponding to the battery capacity. In an embodiment, the capacity indicating portion 425 may indicate an interval corresponding to the battery capacity based on at least one of an on/off of the at least one LED element, a color of output light of the LED element, and blinking of output light thereof.

For example, the capacity indicating portion 425 may include a plurality of LED elements. FIG. 5 illustrates a case where the capacity indicating portion 425 includes three LED elements.

In an embodiment, the scanner 110 may receive battery information from the battery 140. In an embodiment, the battery information may include at least one of data indicating a battery capacity and data indicating a battery temperature. In an embodiment, the data indicating the battery capacity may include at least one of information indicating the maximum charge capacity of the battery, a current charge capacity remaining in the battery, a battery voltage, a battery life, etc.

In an embodiment, when the connector of the battery 140 is connected to the connector of the scanner 110, the battery 140 may transmit battery information to the scanner 110. In an embodiment, the scanner 110 may receive the battery information from the battery 140 and identify a battery capacity based on the battery information.

In an embodiment, the scanner 140 may output the battery capacity by using one or more LED elements included in the capacity indicating portion 425. In an embodiment, the scanner 140 may indicate the battery capacity by controlling at least one of an on/off, color, and blinking of the one or more LED elements included in the capacity indicating portion 425.

For example, when the battery capacity is greater than or equal to a certain reference value, e.g., 70%, relative to the maximum charge capacity, the scanner 110 may control all of the three LED elements to emit blue light. The scanner 110 may control the LED elements to turn off one by one as the battery capacity decreases. The scanner 110 may control only one of the three LED elements to turn on when the battery capacity is less than or equal to a reference value, e.g., 20%, relative to the maximum charge capacity. When the battery capacity drops to, for example, 10% or lower, the scanner 110 may control all of the three LED elements to turn off, control all of the three LED elements to output green light, control all of the three LED elements to output blinking green light, or control two of the three LED elements to turn off and only one LED element to output blinking green light. However, this is only an embodiment, and the present disclosure is not limited thereto.

The user may easily identify a level of the battery capacity through an on/off, color, blinking, etc. of the LED elements included in the capacity indicating portion 425. When it is determined that the power is likely to be turned off due to a low battery capacity, the user may finish a scanning operation being performed, press a power button provided in the user input unit to turn off the scanner 110, and then perform an operation such as replacing the battery 140 with another charged battery 140, replacing it with the dummy cable 330, or the like. Therefore, problems such as the scanner 110 suddenly turning off due to a low charging capacity of the battery 140 may not occur.

In an embodiment, the battery 140 may be in a form separable into a plurality of batteries, such as two partial batteries. The battery 140 may be in a form in which two partial batteries such as a first partial battery and a second partial battery are joined together using a magnet or the like. In an embodiment, while the battery 140 is inserted into the scanner 110 to supply power to the scanner 110, when the battery capacity becomes significantly low, the user may divide the battery 140 into two partial batteries, leave the first partial battery inserted into the scanner 110 as is, and replace the second partial battery with another charged partial battery, so that power may be supplied to the scanner 110.

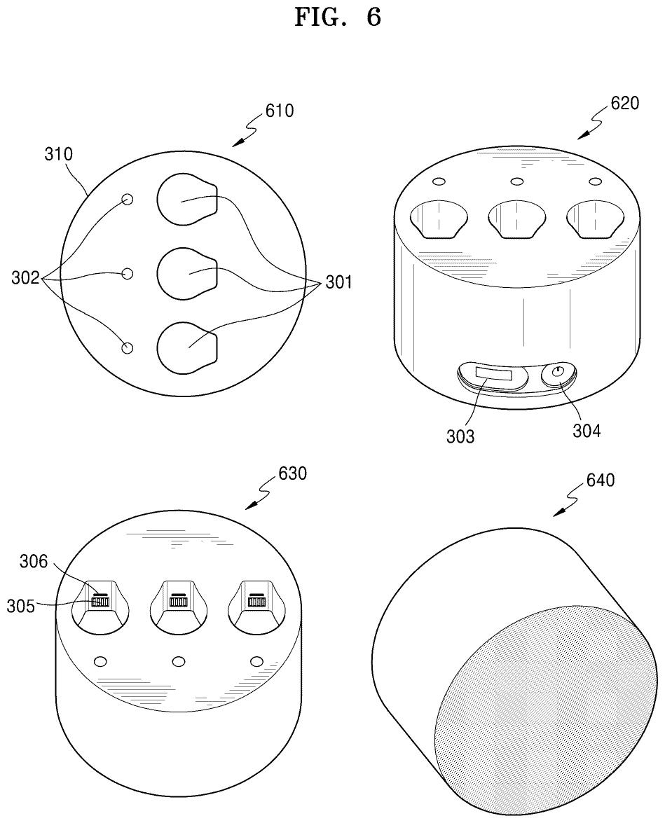

FIG. 6 is a diagram illustrating a charger 310 according to an embodiment.

The drawing reference numeral 610 of FIG. 6 represents a top view of the charger 310.

In an embodiment, the charger 310 may be inserted into the scanner 110 in a detachable form to charge the battery 140 that supplies power to the scanner 110.

In an embodiment, the charger 310 may include an insertion portion into which the battery 140 is to be inserted. In an embodiment, the insertion portion included in the charger 310 and into which the battery 140 is inserted is referred to as a second insertion portion.

As illustrated in the drawing with the drawing reference numeral 610, the charger 310 may have a second insertion portion 301 formed therein. In an embodiment, the second insertion portion 301 may be a space having a concave shape that is recessed from the top to the bottom of the charger 301.

In an embodiment, the second insertion portion 301 may have a shape corresponding to a shape of the battery 140. For example, an innermost region of the second insertion portion 301 that is concavely recessed may have the same shape as one side of a predetermined region of the battery 140 that is in contact with the second insertion portion 301. For example, when one side of the first region of the battery 140 contacts the second insertion portion 301 and has one end of a flat shape and the remaining portion of a round shape, the innermost region of the second insertion portion 301 may also be formed to have one end of a flat shape and the remaining portion of a round shape.

In an embodiment, a magnet may be provided in the innermost region of the second insertion portion 301 that is concavely recessed. In an embodiment, the magnet included in the second insertion portion 301 may be combined with a metal provided on the one side of the first region of the battery 140, thereby allowing the battery 140 to be fixedly attached to the charger 310.

In an embodiment, the charger 310 may include a plurality of second insertion portions 301. For example, as illustrated in the drawing with the drawing reference numeral 610, the charger 310 may include three second insertion portions 301. The battery 140 may be inserted into each of the three second insertion portions 301. However, this is only an embodiment, and the number of second insertion portions 301 included in the charger 310 may be changed in various ways.

In an embodiment, the charger 310 may include a charge state indicating portion 302. In an embodiment, the charge state indicating portion 302 may be located near the second insertion portion 302.

In an embodiment, the charge state indicating portion 302 may include an LED element. The LED element included in the charge state indicating portion 302 may indicate a state of charge of the battery 140 inserted into the second insertion portion 301.

In an embodiment, the charger 310 may include as many charge state indicating portions 302 as there are the second insertion portions 301. For example, as illustrated in FIG. 6, the charger 310 may include three LED elements. Each of the three LED elements may independently indicate a state of charge of each of the three batteries 140 inserted into each of the three second insertion portions 301.

In an embodiment, the state of charge of the battery 140 may include at least one of a state in which the battery 140 is not inserted, a state in which the battery 140 is being charged, a state in which charging of the battery 140 is complete, and a state in which an error such as a poor connection has occurred.

In an embodiment, the charge state indicating portion 302 may change an on/off of the LED element differently, change a color of light emitted by an LED element, adjust an interval at which the LED element blinks differently, or the like, depending on the state of charge of the battery 140. For example, the charge state indicating portion 302 may turn off an LED element when the battery 140 is not inserted into the second insertion portion 301, and may turn on the LED element when the battery 140 is inserted thereinto. For example, the charge state indicating portion 302 may cause the LED element to emit blinking blue light when the battery 140 inserted into the second insertion portion 301 is being charged, and cause the LED element to continuously emit steady blue light when charging is complete. For example, the charge state indicating portion 302 may cause the LED element to emit blinking green light when a connection error occurs, such as when the battery 140 is not properly inserted into the second insertion portion 301.

However, this is only an embodiment, and the charge state indicating portion 302 may change an operation, color, etc. of an LED element in various ways depending on the state of charge of the battery 140.

The drawing reference numeral 620 represents a perspective view of the charger 310 from one side. Referring to the drawing reference numeral 620, a power port may be included on a side of the charger 310. The power port may include a first connection port 304 to which an adapter port is connected and a second connection port 303 to which the connection portion of the dummy cable 330 is connected.

In an embodiment, the adapter port is connected to the first connection port 304 so that power may be supplied from the adapter 320. The charger 310 may charge the battery 140 inserted into the second insertion portion 301 with power supplied from the adapter 320.

In an embodiment, the connection portion of the dummy cable 330 may be connected to the second connection port 303. When the connection portion of the dummy cable 320 is connected to the second connection port 303 while the dummy portion of the dummy cable 320 is inserted into the scanner 110, the charger 310 may supply power to the second connection port 303 so that the power may be supplied to the scanner 110 via the dummy cable 330.

The drawing reference numeral 630 represents a perspective view of the charger 310 from another side. The other side of the charger 310 shown by the drawing reference numeral 630 may be in an opposite direction to the one side of the charger 310 shown by the drawing reference numeral 620.

In an embodiment, the second insertion portion 301 may include one of a protrusion and a groove. For example, as illustrated by the drawing reference numeral 630, a groove 306 may be formed in the second insertion portion 301. The groove 306 formed in the second insertion portion 301 may be used to engage with a protrusion provided in the first region of the battery 140.

In an embodiment, the second insertion portion 301 may include a terminal. In an embodiment, the terminal provided in the second insertion portion 301 is referred to as a third connector 305.

In an embodiment, the third connector 305 may be connected to the first connector provided in the battery 140 and used to charge the battery 140.

The drawing reference numeral 640 represents a drawing of the bottom of the charger 310 as viewed from a side. Referring to the drawing reference numeral 640, a pad may be attached to a bottom of the charger 310. The pad may be attached to the bottom of the charger 310 to prevent the charger 310 from sliding.

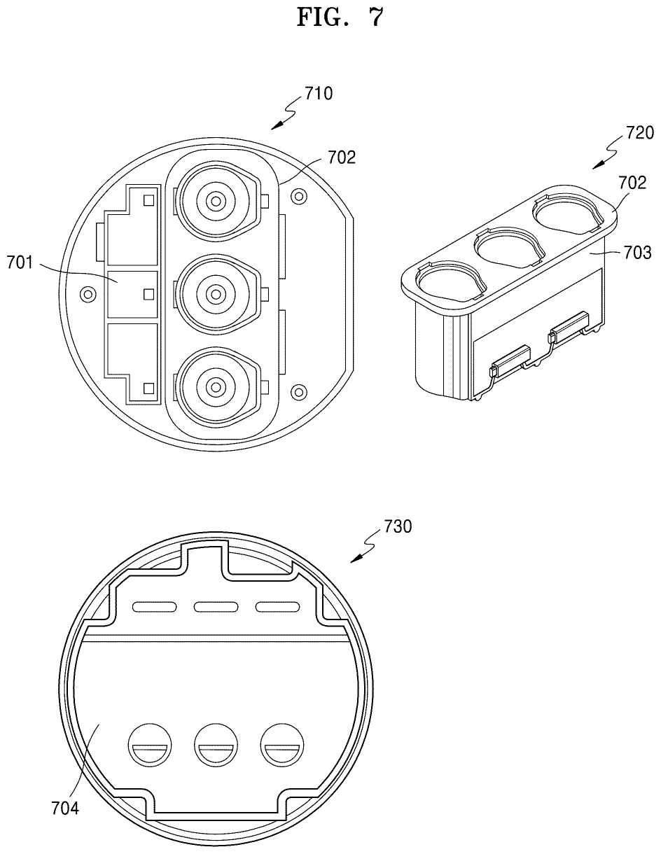

FIG. 7 is a diagram illustrating the interior of the charger 310 according to an embodiment.

The drawing reference numeral 710 of FIG. 7 represents a view of the interior of the charger 310 as viewed from the top. The top view of reference numeral 710 shows the charger 310 with a top case removed from the charger 310 shown in the top view of the drawing reference numeral 610 of FIG. 6.

In an embodiment, the charger 310 may be equipped with a printed circuit board (PCB). A PCB may refer to a circuit board that connects and fixes electronic component terminals to the inside of a main body of the charger 310. A PCB may be a circuit board on which various electronic devices/components such as ICs, resistors, etc. are collected, arranged, and mounted on the board, and are connected via conductive connection paths (patterns) formed by processing copper (Cu).

The drawing with the drawing reference numeral 710 illustrates an insertion portion case 702 and a first PCB attached to one side of the insertion portion case 702. The insertion portion case 702 may be a case for accommodating the second insertion portion. The first PCB may include an LED element for indicating a state of charge. When the number of second insertion portions 301 included in the charger 310 is three, the first PCB may be provided with three LED elements each independently indicating the state of charge of each of the batteries 140 inserted into the second insertion portions 301.

The drawing reference numeral 720 represents a drawing of the insertion portion case 702 shown by the drawing reference numeral 710 as viewed from a side. A second PCB 703 may be attached to a side opposite to the one side of the insertion portion case 702. The third connector 305 may be disposed on the second PCB 703. The third connector 305 may be electrically connected to the first connector 144 provided in the battery 140.

In an embodiment, the third connector 305 and the first connector 144 may each include a plurality of pins. For example, the third connector 305 and the first connector 144 may each include four pins. Two of the four pins may be pins for power control, and the remaining two pins may be pins for data communication. The two pins for power control may be a power pin and a ground pin. In addition, the two pins for data communication may be a pin for transmitting data about a battery capacity and a pin for transmitting data about a battery temperature, respectively.

In an embodiment, the pins included in the third connector 305 and the first connector 144 may be arranged so that corresponding pins are connected to each other. For example, the plurality of pins included in the third connector 305 and the first connector 144 may be arranged so that the pins for power control are interlocked with each other and the pins for data communication are interlocked with each other.

The drawing reference numeral 730 represents a drawing illustrating a third PCB 704 arranged at a bottom of the insertion case 702 illustrated by the drawing reference numeral 710. The third PCB 704 may be connected to the first PCB and the second PCB via wires.

In an embodiment, a charger IC chip may be mounted on the third PCB 704. In an embodiment, the charger IC chip may control a charging operation for the battery 140 inserted into the second insertion portion 301.

In an embodiment, the third PCB 704 may be provided with as many charger IC chips as there are second insertion portions 301 into which the batteries 140 may be inserted. For example, when there are three second insertion portions 301, the third PCB 704 may be provided with three charger IC chips. Each of the three charger IC chips may control a charging operation for each of the three batteries 140 inserted into the three second insertion portions 301.

In an embodiment, the charger IC chip mounted on the third PCB 704 may control operation of the LED element mounted on the first PCB 701 so that the LED element may indicate the state of charge of the battery 140.

In an embodiment, in response to the first connector 144 of the battery 140 being connected to the third connector 305 provided on the second PCB 703, the charger IC chip may identify the battery capacity and the battery temperature via the third connector 305 and the first connector 144.

The battery 140 may include an IC chip to obtain battery information. The battery information may include at least one of data indicating a battery capacity and data indicating a battery temperature. The battery 140 is equipped with a temperature sensor for sensing a battery temperature. The IC chip included in the battery 140 may obtain data indicating a battery temperature by using the temperature sensor.

In an embodiment, the battery 140 may transmit the data indicating the battery temperature to the charger 310 via the first connector 144 and the third connector 305.

In an embodiment, the charger IC chip may read a battery capacity via the first connector 144 and the third connector 305. For example, the charger IC chip may read a voltage value and a current value of the battery 140 via the pins for power control included in the third connector 305 and the first connector 144. By doing so, the charger IC chip may identify how much charge capacity remains in the battery 140.

In an embodiment, the charger IC chip may determine a charge voltage and a charge current, based on the battery capacity and the battery temperature. In an embodiment, the charger IC chip may charge the battery 140 by providing the determined charge voltage and charge current to the battery 140 via the pins for power control included in the third connector 305 and the first connector 144.

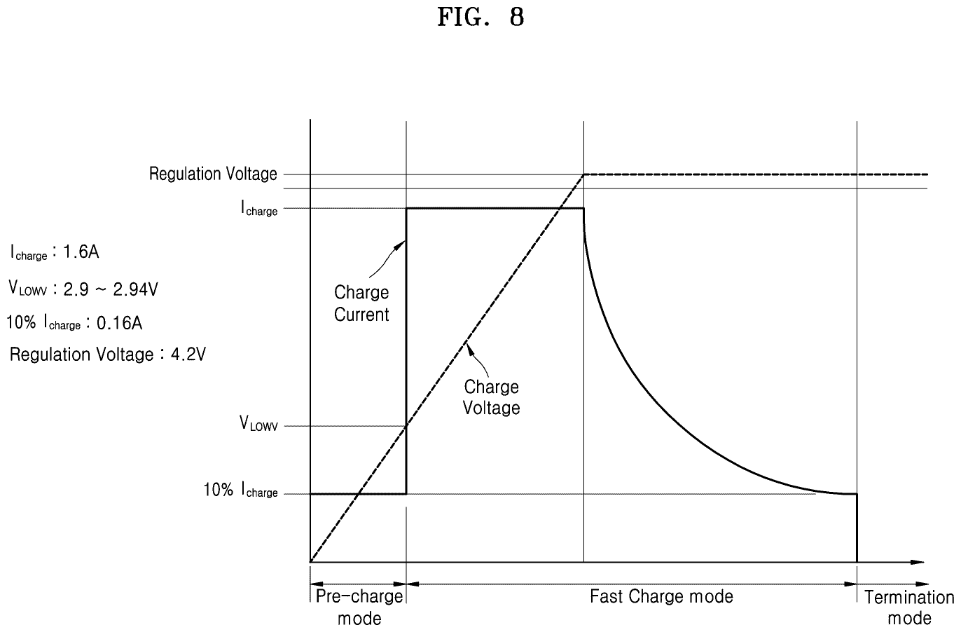

FIG. 8 is a graph showing a charge voltage and a charge current according to an embodiment.

The graph in FIG. 8 shows time on the horizontal axis and a charge voltage and a charge current on the vertical axis. In the graph, a solid line represents a charge current, and a dashed line represents a charge voltage.

In an embodiment, when the battery 140 is inserted into the charger 310, the connectors of the battery 140 and the charger 310 are connected to each other. That is, the first connector provided in the battery 140 and the third connector provided in the charger 310 are connected. In an embodiment, the charger IC chip included in the charger 310 may identify that the battery 140 is inserted into the charger 310 in response to the first connector being connected to the third connector.

In an embodiment, the charger IC chip may identify a voltage and a current of the battery 140 via the pins for power control included in the first connector and the third connector. In an embodiment, the charger IC chip may identify a battery capacity from the voltage and current of the battery 140.

In an embodiment, the charger IC chip may automatically select a charge mode based on the battery capacity and perform a charging operation.

In an embodiment, charge modes may include a pre-charge mode, a fast charge mode, and a termination mode.

The graph of FIG. 8 shows, from left to right, voltages and currents applied to the battery 140 in pre-charge mode, fast charge mode, and termination mode, respectively.

First, the pre-charge mode may be a mode that operates when a battery voltage is about 2.0 V or lower. In the pre-charge mode, the battery 140 may be charged with a constant current, e.g., 0.16 amperes (A), which is 10% of the charge current when a maximum charge current I_{charge} is 1.6 A. In the pre-charge mode, a battery voltage may be allowed to rise to a certain level V_{LOWV}, e.g., about 2.90 V to 2.94 V or higher.

In an embodiment, the fast charge mode may be a mode that operates when the battery voltage is above a certain level V_{LOWV}, e.g., about 2.9 V to 2.94 V or higher. In this mode, the battery may be charged at the maximum charge current I_{charge}, e.g., 1.6 A, to complete charging quickly. In this mode, the battery voltage may rise to reach a certain level (regulation voltage), e.g., 4.2 V. Also, in this mode, the current gradually decreases as the battery voltage increases.

In an embodiment, after the charging of the battery 140 is completed, the charge mode may be switched to the termination mode. In the termination mode, the charge current is zero (0) A, and the battery 140 is protected from overcharging.

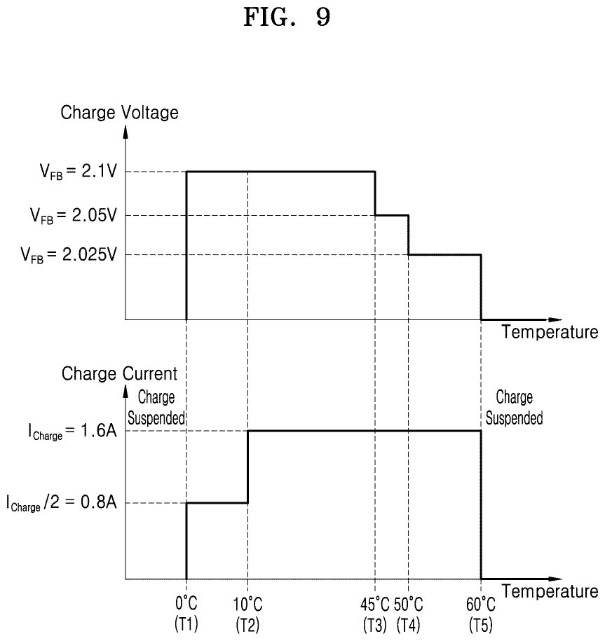

FIG. 9 is graphs illustrating the charger 310 adjusting a charge voltage and a charge current by taking into account a battery temperature, according to an embodiment.

In an embodiment, the charger 310 may adjust a charge voltage and a charge current by considering a battery temperature when charging the battery 140 in the fast charge mode.

In an embodiment, the charger 310 may set a maximum charge current value for the battery 140 and charge the battery 140 at that value.

For example, in an embodiment, the charger 310 may use a method of charging the batteries 140 by setting a maximum value of the charge current for each battery, rather than charging the batteries 140 by distributing a set amount of power when charging the batteries. When charging is performed in this manner, the charging time should be the same regardless of the number of batteries 140 being charged. However, unlike a case where only one battery 140 is inserted and charged in the charger 310, when a plurality of batteries 140 are inserted and charged, there is a possibility that the batteries 140 and the charger 310 may deteriorate due to a rise in the battery temperature.

Accordingly, in an embodiment, the charger 310 may control the charging operation for the battery 140 by taking into account the battery temperature. In an embodiment, to prevent deterioration of the battery 140, the charger 310 may check for a threshold temperature and change a maximum charge current value to a value that is lower than an original value.

In an embodiment, each battery 140 may have an IC chip embedded therein. In addition, a temperature sensor may be built into the IC chip to obtain a temperature in real time. The IC chip in the battery 140 may transmit in real time data indicating a battery temperature to the charger IC chip of the charger 310 via the connector.

In an embodiment, the charger IC chip of the charger 310 may receive the data indicating the battery temperature from the battery 140. When the charger 310 charges the plurality of batteries 140, the internal temperature of each battery 140 itself increases due to heat generated by the batteries 140 and the charger 310. In this case, the charger IC chip receives data indicating a higher battery temperature from the battery 140.

In general, a temperature rises when power increases. Because the power is determined by the product of a voltage and a current, when one of the voltage and the current is lowered, the power decreases and the battery temperature also decreases accordingly.

In an embodiment, the charger IC chip of the charger 310 may determine a charge voltage and a charge current differently according to a temperature range, as shown in the graphs of FIG. 9.

In an embodiment, in upper and lower graphs illustrated in FIG. 9, horizontal axes represent a battery temperature, and vertical axes represent a charge voltage and a charge current, respectively.

In an embodiment, the charger IC chip performs charging for the battery 140 only in an interval where the temperature is T1 to T5 (0° C. to 60° C.), and stops charging when the charger IC chip reads a temperature value that falls outside the interval.

Referring to the graphs of FIG. 9, the charger IC chip may apply only a current corresponding to half of a maximum charge current and a voltage equal to a maximum charge voltage in an interval where the temperature is T1 to T2 (0° C. to 10° C.). Furthermore, the charger IC chip may apply the maximum charge current and the maximum charge voltage in an interval where the temperature is T2 to T3 (10° C. to 45° C.). Furthermore, the charger IC chip may apply the maximum charge current and a voltage lower than the maximum charge voltage in an interval where the temperature is T3 to T4 (45° C. to 50° C.). In addition, in an interval where the temperature is T4 to T5 (50° C. to 60° C.), the charger IC chip may apply the maximum charge current and a voltage that is lower than the voltage applied in the interval when the temperature is T3 to T4.

In an embodiment, the charger IC chip may apply a charge voltage and a charge current to the battery 140 variably according to a temperature, as shown in the graphs of FIG. 9, thereby preventing the battery 140 and charger 310 from deteriorating.

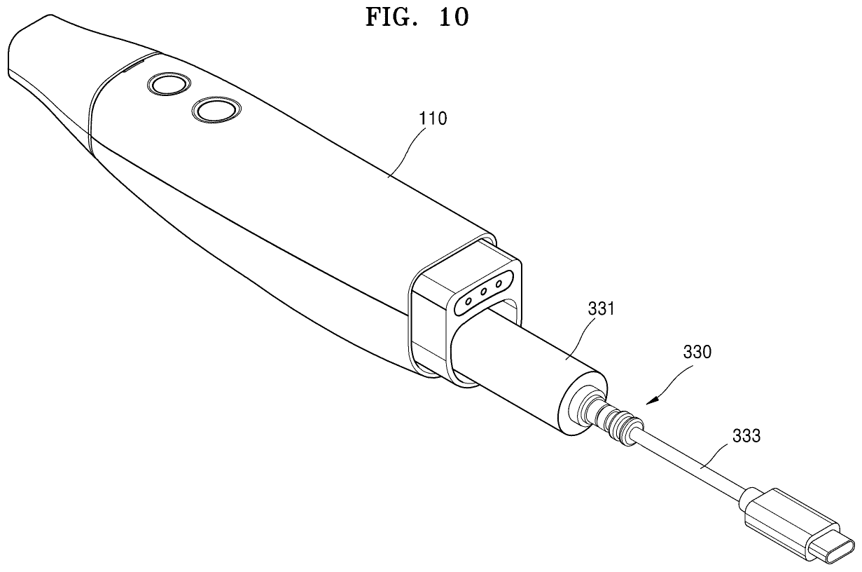

FIG. 10 is a diagram illustrating the dummy cable 330 being inserted into the scanner 110, according to an embodiment.

Referring to FIG. 10, in an embodiment, the dummy cable 330 may be inserted into the scanner 110. In an embodiment, the scanner 110 may receive power from the charger 310 via the dummy cable 330. For example, in a situation where the scanner 110 is not able to receive power from the battery 140, such as when the battery 140 is not charged or the battery 140 is lost, the scanner 110 may receive power via the dummy cable 330 instead of the battery 140.

In an embodiment, the dummy cable 330 may be a power supply device in the form of a cable used instead of the battery 140. The dummy cable 330 may supply power to the scanner 110 via a cable. In an embodiment, the dummy cable 330 may include a dummy portion 331 having the shape of a battery and a connection portion 333 connected to the charger 310. A region of the dummy portion 331 that is inserted into the scanner 110 may have the same shape as the first region of the battery 140. Therefore, like the battery 140, the dummy portion 331 may be easily inserted into the first insertion portion of the scanner 110.

In an embodiment, the dummy portion 331 may include a terminal. The terminal included in the dummy portion 331 is referred to as a fourth connector.

In an embodiment, the fourth connector may be connected to the second connector provided in the scanner 110. In an embodiment, the fourth connector may include the same number of pins as the first connector provided in the battery 140. For example, when the first connector provided in the battery 140 includes four pins, the fourth connector may also include four pins. Two of the four pins may be pins for power control, and the remaining two pins may be pins for data communication.

In an embodiment, the fourth connector may transmit information indicating that the dummy cable 330 is a dummy to the second connector via a pin for data communication.

The dummy portion 331 may be connected to the charger 310 via the connection portion 333.

In an embodiment, the dummy portion 331 may receive power from the charger 310 via the connection portion 333 and provide the power to the scanner 140 via pins for power control included in the second connector and the fourth connector.

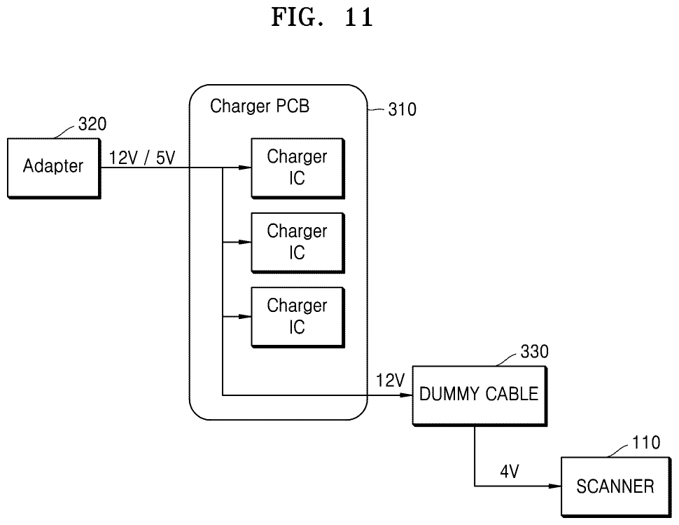

FIG. 11 is a power system diagram illustrating a power supply device for a scanner, according to an embodiment.

Referring to FIG. 11, in an embodiment, the charger 310 may be connected to the adapter 320. The adapter 320 may be connected to a power port provided on the charger 310 via a connection cable. The adapter 320 may convert an alternating current from a power system into a direct current and supply power to the charger 310. For example, the adapter 320 may supply power (12 V, 5 A) to the charger 310.

In an embodiment, the charger 310 may be equipped with a plurality of charger IC chips. The plurality of charger IC chips may each charge a corresponding battery with a maximum charge current, e.g., 1.6 A.

In an embodiment, the dummy cable 330 may also be connected to the charger 310. When the dummy cable 330 is connected to the power port provided on the charger 310, the charger 310 may charge a plurality of batteries while simultaneously supplying power to the dummy cable 330.

In an embodiment, the charger 310 may input a voltage of 12 V provided by the adapter 320 to the dummy cable 330. The dummy cable 330 may regulate the voltage input from the charger 310 to a voltage suitable for the scanner 110, e.g., 4 V, and provide the regulated voltage to the scanner 110.

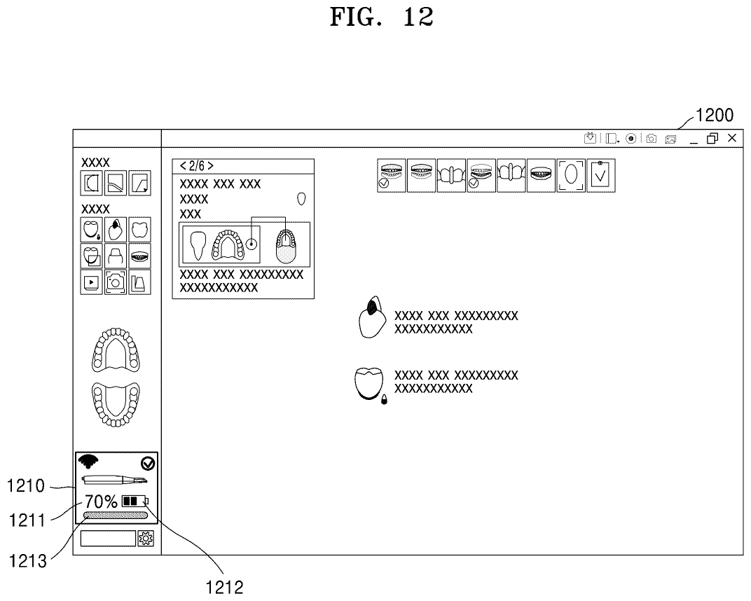

FIG. 12 is a diagram illustrating the data processing device 120 outputting an interface screen showing a battery capacity, according to an embodiment. Referring to FIG. 12, the data processing device 120 may receive battery information from the scanner 110. In an embodiment, the battery information may include data indicating a battery capacity. The data indicating the battery capacity may include at least one of the maximum charge capacity of the battery, information about what percentage of capacity currently remains in the battery, a battery voltage, and a battery life.

In an embodiment, in response to receiving the battery information, the data processing device 120 may output an interface screen 1210 indicating a battery capacity on a display 1200. In an embodiment, the interface screen 1210 indicating the battery capacity may include information indicating a current charge capacity of the battery 140 relative to the maximum charge capacity of the battery 140 by using at least one of letters, pictures, numbers, and colors.

In an embodiment, the interface screen 1210 indicating the battery capacity may be output on a portion of a display 1200. The size, output location, transparency, and/or shape of the interface screen 1210 indicating the battery capacity may be variously modified. In addition, the letters, pictures, numbers, colors, etc. used to indicate the battery charge capacity, which is included in the interface screen 1210 indicating the battery capacity, may be variously modified.

The user may easily identify a charge capacity of the battery by viewing the interface screen 1210 indicating a battery capacity, which is output on the display 1200 of the data processing device 120.

For example, the interface screen 1210 indicating the battery capacity illustrated in FIG. 12 may include a number 1211 indicating that the battery capacity is 70% of the maximum charge capacity, and a picture 1212 indicating a state of charge of the battery.

Furthermore, the interface screen 1210 indicating the battery capacity may also use a color to represent the current charge capacity of the battery. For example, the number 1211, the picture 1212, etc. included in the interface screen 1210 indicating the battery capacity may all be displayed in blue when the battery capacity is 60% or more, and may all be displayed in light green when the battery capacity is at least 20% but less than 60%. Furthermore, when the battery capacity is less than 20%, the number 1211, the picture 1212, etc. included in the interface screen 1210 indicating the battery capacity may be displayed in green.

Furthermore, the interface screen 1210 indicating the battery capacity may include a bar graph 1213 having a length corresponding to the battery capacity. The bar graph 1213 may be a graph that expresses the battery capacity as the length of a straight line. For example, the bar graph 1213 may have a maximum length when the battery capacity is 70% or more, a length that is half the maximum length when the battery capacity is at least 30% but less than 70%, and a minimum length when the battery capacity is less than 30%.

In addition, at least one of the number 1211, the picture 1212, and the bar graph 1213 included in the interface screen 1210 indicating the battery capacity may blink when the battery capacity is below a certain reference value, e.g., less than 10%.

The user may easily identify the charge capacity of the battery 140 by viewing the interface screen 1210 indicating the battery capacity, which is output on the display 1200 of the data processing device 120.

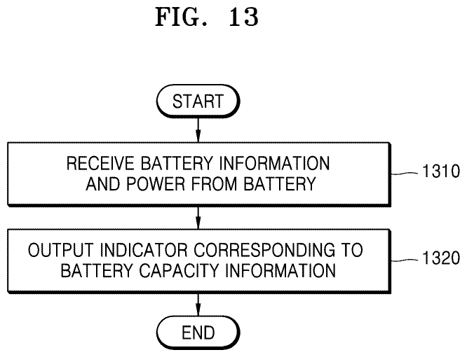

FIG. 13 is a flowchart illustrating an operation in which the scanner 110 receives power from the battery 140, according to an embodiment.

In an embodiment, the battery 140 may be inserted into the scanner 110 in a detachable form.

In an embodiment, when the battery 140 is inserted into the scanner 110, the first connector provided in the battery 140 may be connected to the second connector provided in the scanner 110.

In an embodiment, the scanner 110 may receive battery information and power from the battery 140 (operation 1310). In an embodiment, the scanner 110 may receive the battery information and the power from the battery 140 via the first connector and the second connector.

In an embodiment, the scanner 110 may identify battery capacity information from the battery information.

In an embodiment, the scanner 110 may output an indicator corresponding to battery capacity information (operation 1320).

In an embodiment, the scanner 110 may output an indicator corresponding to battery capacity information by controlling at least one of an on/off of at least one LED element, a color of output light of the LED element, and blinking of output light thereof according to the battery capacity information.

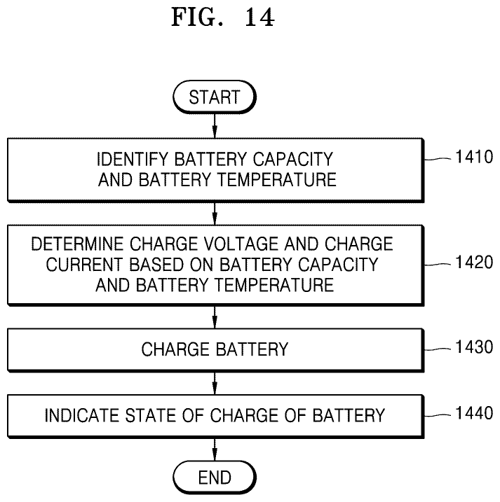

FIG. 14 is a flowchart illustrating an operation in which the charger 310 charges the battery 140, according to an embodiment.

In an embodiment, when the battery 140 is inserted into the charger 310, the first connector provided in the battery 140 may be connected to the third connector provided in the charger 310.

In an embodiment, the charger 310 may identify a battery capacity and a battery temperature (operation 1410).

In an embodiment, the charger 310 may identify a capacity of the battery 140 in response to the first connector being connected to the third connector. Furthermore, in an embodiment, the charger 310 may receive data indicating a battery temperature from the battery 140 via the first connector and the third connector.

In an embodiment, the charger 310 may determine a charge voltage and a charge current, based on the battery capacity and the battery temperature (operation 1420).

In an embodiment, the charger 310 may charge the battery 140 (operation 1430). In an embodiment, the charger 310 may charge the battery 140 by providing the determined charge voltage and charge current to the battery 140 via the first connector and the third connector.

In an embodiment, the charger 310 may indicate a state of charge of the battery 140 (operation 1440).

In an embodiment, the charger 310 may use one LED element for each battery 140 to indicate a state of charge of each battery 140 by using at least one of an on/off, color, and blinking of the LED element.

The operation of a charger or scanner according to an embodiment of the present disclosure may be implemented in the form of program commands executable by various types of computers and recorded on computer-readable media. Furthermore, according to an embodiment of the present disclosure, a computer-readable storage medium having recorded thereon one or more programs including at least one instruction for executing a charging operation may be provided.

Furthermore, an operation method of a charger according to an embodiment of the present disclosure may be embodied in a computer program product including a computer-readable recording medium having recorded thereon a program for implementing the operation method of the charger, which includes identifying a battery capacity and a battery temperature in response to connectors of the charger and a battery being connected to each other, determining a charge voltage and a charge current based on the battery capacity and the battery temperature, charging the battery by providing the determined charge voltage and charge current to the battery, and indicating a state of charge of the battery.

The computer-readable storage medium may include program commands, data files, data structures, etc. either alone or in combination. Examples of the computer-readable storage medium may include magnetic media such as hard disks, floppy disks, and magnetic tapes, optical media such as a compact disk read-only memory (CD-ROM) and digital versatile disks (DVDs), magneto-optical media such as floptical disks, and hardware devices that are configured to store and perform program commands, such as ROM, random access memory (RAM), flash memory, etc.

Here, a machine-readable storage medium may be provided in the form of a non-transitory storage medium. In this regard, the term ‘non-transitory storage medium’ may mean that the storage medium is a tangible device. Furthermore, the ‘non-transitory storage medium’ may include a buffer for temporarily storing data.

According to an embodiment, operation methods of a scanner or a charger according to various embodiments of the present disclosure may be included in a computer program product when provided. The computer program product may be distributed in the form of a machine-readable storage medium (e.g., CD-ROM). Alternatively, the computer program product may be distributed (e.g., downloaded or uploaded) on-line via an application store (e.g., Google™M, Play Store TM, etc.) or directly between two user devices (e.g., smartphones). Specifically, the computer program product according to the disclosed embodiment may include a storage medium having recorded thereon a program including at least one instruction for performing the operation method of the scanner or charger according to the disclosed embodiment.

While embodiments have been particularly described above, the scope of the present disclosure is not limited thereto, and various modifications and improvements made by those skilled in the art using a basic concept of the present disclosure also fall within the scope of the present disclosure as defined in the following claims.

1. A scanner comprising:
a first insertion portion into which a battery is to inserted in a detachable form;
a capacity indicating portion; and
one or more processors configured to execute one or more instructions,
wherein the one or more processors are configured to execute the one or more instructions to,
in response to a first connector provided in the battery being connected to a second connector provided in the first insertion portion, receive battery information and power from the battery via the first connector and the second connector, and
output an indicator corresponding to the battery information via the capacity indicating portion.

2. The scanner of claim 1, wherein
the battery information comprises a battery capacity and a battery temperature, and
the indicator corresponding to the battery information indicates the battery capacity.

3. The scanner of claim 1, wherein
the capacity indicating portion comprises at least one light-emitting diode (LED) element, and
the indicator corresponding to the battery information indicates an interval corresponding to the battery information based on at least one of an on/off of the at least one LED element, a color of output light of the at least one LED element, and blinking of output light thereof.

4. The scanner of claim 1, wherein
the battery comprises at least one of one of a protrusion and a groove and a metal that adheres to a magnet, and
the first insertion portion comprises at least one of one of a groove and a protrusion for engaging with the protrusion or the groove in the battery and a magnet, and is coupled with the battery by using the at least one of the one of the groove and the protrusion and the magnet.

5. The scanner of claim 1, wherein each of a portion of the battery inserted into the first insertion portion and the first insertion portion comprises a round region and an angular region, and the first connector and the second connector are respectively provided in the angular regions of the portion of the battery and the first insertion portion.

6. The scanner of claim 1, wherein, in response to a dummy cable, which is not the battery, being inserted into the first insertion portion, the scanner receives power from a charger via the dummy cable,
the dummy cable comprises a dummy portion including a fourth connector and inserted into the first insertion portion and a connection portion connected to the charger, and
the one or more processors are further configured to execute the one or more instructions to receive power from the charger via the second connector and the fourth connector.

7. The scanner of claim 1, further comprising an optical unit, wherein the one or more processors are further configured to execute the one or more instructions to, in response to receiving the power from the battery, control the optical unit to obtain scan data regarding an object, and generate a three-dimensional oral cavity model for the object based on the scan data regarding the object.

8. A charger for charging a battery that is inserted into a scanner in a detachable form and supplies power to the scanner, the charger comprising:
a second insertion portion into which the battery is inserted and provided with a third connector;
a charger integrated circuit (IC) chip connected to the third connector via a printed circuit board (PCB); and
a state indicating portion configured to indicate a state of charge of the battery,
wherein the charger IC chip is configured to,
in response to the third connector being connected to a first connector of the battery, identify a battery capacity and a battery temperature via the third connector and the first connector,
determine a charge voltage and a charge current based on the battery capacity and the battery temperature,
charge the battery by providing the determined charge voltage and charge current to the battery via the third connector and the first connector, and indicate a state of charge of the battery via the state indicating portion.

9. The charger of claim 8, wherein the state of charge of the battery comprises at least one of a state in which the battery is not inserted, a state in which the battery is being charged, a state in which charging is complete, and a state in which an error has occurred.

10. The charger of claim 8, wherein
the second insertion portion comprises a plurality of insertion spaces into which a plurality of batteries are to be independently inserted,
the charger IC chip is included as a plurality of charger IC chips, wherein the number of the plurality of charger IC chips is equal to the number of the plurality of insertion spaces, to control charging of the plurality of batteries respectively inserted into the plurality of insertion spaces, and
the state indicating portion comprises a plurality of light-emitting diodes (LEDs) in a number that is as many as the number of the plurality of insertion spaces, each of the plurality of LED elements independently indicating a state of charge of each of the plurality of batteries respectively inserted into the plurality of insertion spaces.

11. The charger of claim 10, wherein a first charger IC chip among the plurality of charger IC chips is configured to identify a first battery capacity and a first battery temperature for a first battery inserted into a first insertion space among the plurality of insertion spaces, determine, based on the first battery capacity and the first battery temperature, a first charge voltage and a first charge current to be provided to the first battery, and provide the determined first charge voltage and first charge current to the first battery.

12. The charger of claim 8, further comprising a connection port, wherein, in response to the other end of a dummy cable, of which one end is inserted into the scanner, being connected to the connection port, the charger supplies power to the scanner via the dummy cable.

13. A battery that is rechargeable and is inserted into a scanner in a detachable form to provide power to the scanner, the battery comprising:
a first connector; and
an integrated circuit (IC) chip,
wherein the IC chip is configured to obtain battery information including data indicating a battery capacity and data indicating a battery temperature, and in response to the first connector being connected to a second connector provided in the scanner, transmit power and the battery information to the scanner via the first connector and the second connector.

14. The battery of claim 13, wherein, in response to the battery being inserted into a second insertion portion included in a charger and the first connector being connected to a third connector provided in the second insertion portion, the IC chip is configured to transmit the battery temperature to the charger via the first connector and the third connector, and the battery is charged by receiving power from the charger.

15. The battery of claim 14, wherein
the battery comprises at least one of one of a protrusion and a groove and a metal that adheres to a magnet,
at least one of the scanner and the charger comprises at least one of one of a groove and a protrusion for engaging with the protrusion or the groove in the battery and a magnet, and
the battery is mounted to the scanner or the charger by using the at least one of the one of the protrusion and the groove and the metal that adheres to the magnet.
